(19) United States
(12) Patent Application Publication
(10) Pub. No.: US 2019/0062818 A1
(43) Pub. Date: Feb. 28, 2019

(54) SENSOR, REAGENT, METHOD FOR MANUFACTURING PROBE MOLECULE, AND METHOD FOR MANUFACTURING POLYMER MOLECULE

(71) Applicant: Kabushiki Kaisha Toshiba, Minato-ku (JP)

(72) Inventors: Hiroko Miki, Kawasaki (JP); Yoshiaki Sugizaki, Fujisawa (JP); Tatsuro Saito, Kawasaki (JP); Atsunobu Isobayashi, Yokohama (JP)

(73) Assignee: Kabushiki Kaisha Toshiba, Minato-ku (JP)

(21) Appl. No.: 15/912,016

(22) Filed: Mar. 5, 2018

(30) Foreign Application Priority Data
Aug. 30, 2017 (JP) 2017-166011

(51) Int. Cl. C12Q 1/6837; C12Q 1/6874; G01N 27/327; G01N 33/487; G01N 27/414; G01N 27/411; G01N 27/407

(52) U.S. Cl. CPC C12Q 1/6837; C12Q 1/6874; G01N 27/3276; G01N 27/4071; G01N 33/48721; G01N 27/4146; G01N 27/4111; G01N 27/3278

(57) ABSTRACT

According to one embodiment, a sensor includes an ionic liquid, a probe molecule, and a sensor element. The probe molecule selectively associates with a designated substance in the ionic liquid. The sensor element detects an association of the probe molecule with the designated substance.

### CROSS-REFERENCE TO RELATED APPLICATION

This application is based upon and claims the benefit of priority from Japanese Patent Application No. 2017-166011, filed on Aug. 30, 2017; the entire contents of which are incorporated herein by reference.

### FIELD

Embodiments described herein relate generally to a sensor, a reagent, a method for manufacturing a probe molecule, and a method for manufacturing a polymer molecule.

### BACKGROUND

In a liquid phase sensing device that detects a target (a target substance) by using a probe configured on a surface of a sensor surface, it is desirable to store the probe in an aqueous solution if the target is a biological molecule. This prevents damage, degradation, and a decrease of the functions of the probe caused by drying of the liquid. However, in a device filled with an aqueous solution, it is difficult to maintain a thin liquid phase due to evaporation of the liquid before use.

### BRIEF DESCRIPTION OF THE DRAWINGS

FIG. 1 is a schematic view of a sensor of an embodiment;

FIG. 2A is a schematic view of a sensor using a sensor element including a graphene film, and FIG. 2B is a schematic view showing a binding between the graphene film and a probe molecule;

FIG. 3 is a schematic view of a sensor using an ionic liquid that is a thin film;

FIG. 4 is a schematic view of a sensor using an ionic liquid that does not have affinity with protein;

FIGS. 5A and 5B are schematic view of a sensor using a blocking agent;

FIG. 6A is a schematic view of a sensor using a blocking agent, FIG. 6B is a schematic view showing Debye length of a sensor using an aqueous solution; FIG. 7 is a schematic view showing the Debye length of a sensor using an ionic liquid;

FIG. 8 is a schematic view showing a method for manufacturing an aptamer (a nucleic acid) by using SELEX;

FIG. 9 is a schematic view showing a method for manufacturing a peptide aptamer by using a two-hybrid method;

FIG. 10 is a schematic view showing a method for manufacturing a probe molecule;

FIGS. 11A to 11D are schematic views showing a method for manufacturing a molecularly imprinted polymer having affinity in an ionic liquid;

FIG. 12 is a schematic view of a reagent utilizing the MB principle;

FIG. 13 is a schematic view of a reagent utilizing the FRET/BRET principle;

FIG. 14 is a schematic view of a reagent utilizing the FRET/BRET principle;

FIGS. 15A and 15B are schematic view of a sensor including a catalyst molecule that promotes a designated chemical reaction in an ionic liquid;

FIG. 16 is a schematic view of a designated substance removal reagent including a catalyst molecule that promotes a designated chemical reaction in an ionic liquid, and a designated substance removal device including the reagent; and

FIG. 17A is a schematic view of a sensor in which a constituent ion of an ionic liquid is embedded as a probe molecule, and FIG. 17B is a structural formula of an example of the ionic liquid used in FIG. 17A.

### DETAILED DESCRIPTION

According to one embodiment, a sensor includes an ionic liquid, a probe molecule, and a sensor element. The probe molecule selectively associates with a designated substance in the ionic liquid. The sensor element detects an association of the probe molecule with the designated substance.

Embodiments are directed to a sensor, a reagent, a method for manufacturing a probe molecule, a method for manufacturing a sensor, and a method for manufacturing a polymer molecule which can prevent deterioration of the function of a probe due to drying of a liquid, and can detect a target over a long period of time, and can maintain detection accuracy provide.

Hereinafter, embodiments will be described with reference to the drawings. Incidentally, in the respective drawings, the same components are denoted by the same reference numerals.

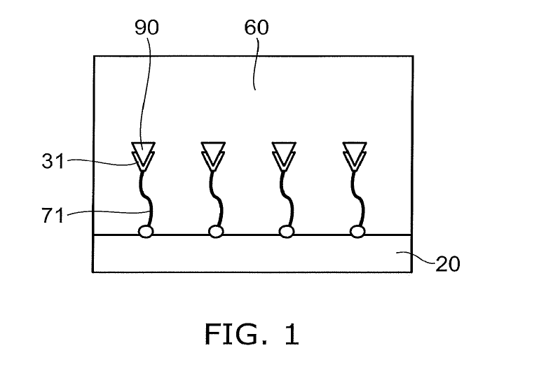

FIG. 1 is a schematic view of a sensor of an embodiment. According to the embodiment, a probe molecule 31 is inside an ionic liquid 60; and a physical quantity (electricity, light, or the like) that is detectable by a sensor element 20 changes due to a designated substance (a target substance) 90 associating with the probe molecule 31.

The probe molecule 31 binds to the surface of the sensor element 20 via a linker 71. The linker 71 adjusts the distance between the probe molecule 31 and the surface of the sensor element 20. The probe molecule 31 binds to one tip of the linker 71.

The other tip of the linker 71 is chemically modified and is chemically bonded or adsorbed to the surface of the sensor element 20. For example, the other tip of the linker 71 is modified with thiol; and the probe molecule 31 can be immobilized at the surface of the sensor element 20 by the thiol binding to Au atoms of the surface of the sensor element 20.

The sensor element 20 is a charge detection element, a surface plasmon resonance element, a SAW (Surface Acoustic Wave) element, a FBAR (Film Bulk Acoustic Resonator) element, a QCM (Quartz Crystal Microbalance) element, or a MEMS (Micro Electro Mechanical System) cantilever element. For example, the surface of the sensor element 20 is formed of gold (Au), graphene, silicon nitride, etc.

The ionic liquid 60 covers the surface of the sensor element 20 and the probe molecule 31 and shields the probe molecule 31 from the external environment (liquid or vapor).

The liquid surface height of the ionic liquid 60 is higher than the upper end height of the probe molecule 31 when referenced to the surface of the sensor element 20. The probe molecule 31 is inside the ionic liquid 60 and contacts the ionic liquid 60.

The probe molecule 31 associates selectively (specifically) with the designated substance (the target substance) 90 in the ionic liquid 60. Then, for example, the sensor element 20 electrically or optically detects the association of the probe molecule 31 with the designated substance (the target substance) 90.

The ionic liquid 60 is a compound including only cations (positive ions) and anions (negative ions), and exists as a liquid in a broad temperature range. In particular, a RTIL (Room

Temperature Ionic Liquid) is a liquid that is in the liquid state near room temperature. For example, an imidazolium cation is an example of the cation. For example, BF₄⁻ and PF₆⁻ are examples of the anion.

The types of the cation and the anion included in the ionic liquid 60 can be selected as necessary. Also, water and/or other liquids may be included in the ionic liquid 60.

The probe molecule 31 includes, for example, at least one selected from the group consisting of an antibody, an aptamer, a peptide aptamer, an enzyme, and a molecularly imprinted polymer.

In the case where the sensor element 20 includes a charge detection element, for example, a charge detection element that includes graphene or carbon nanotubes may be used.

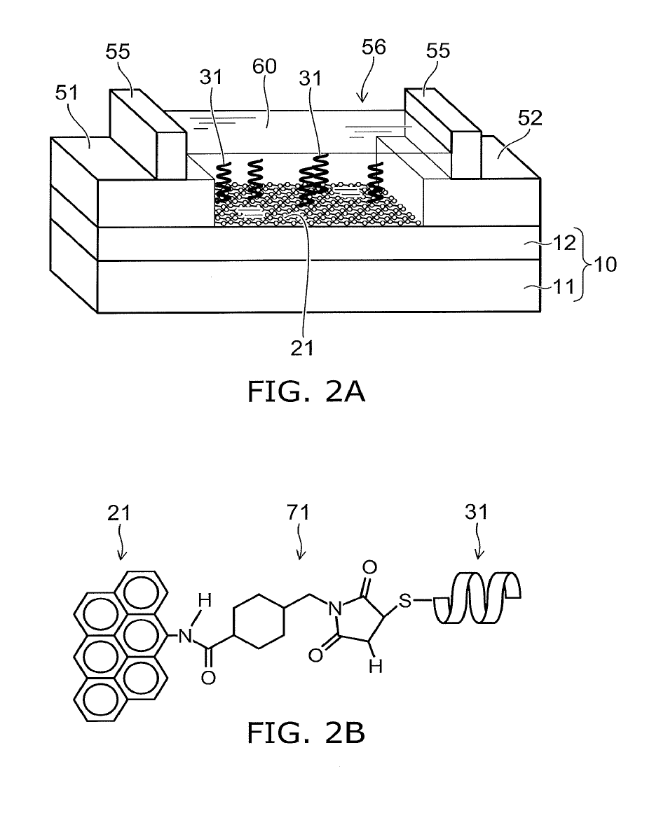

FIG. 2A is a schematic view of a sensor using a sensor element including a graphene film 21.

The sensor includes a foundation 10, the graphene film 21 provided on the foundation 10, and at least two electrodes (a first electrode 51 and a second electrode 52).

For example, the sensor has a FET (field effect transistor) structure. Or, a Wheatstone bridge circuit may be formed in the sensor.

The foundation 10 includes a substrate 11, and a foundation film 12 provided on the substrate 11. The graphene film 21 is provided on the foundation film 12. Or, the graphene film 21 may be provided on the surface of the substrate 11 without providing the foundation film 12. Not-illustrated circuits and transistors also may be formed in the substrate 11.

For example, silicon, silicon oxide, glass, or a high polymer material may be used as the material of the substrate 11. The foundation film 12 is, for example, an insulating film such as a silicon oxide film or a fluorocarbon resin. The foundation film 12 also may have the function of a chemical catalyst for forming the graphene film 21.

The first electrode 51 and the second electrode 52 are provided on the foundation film 12 or on the graphene film 21. The materials of the first electrode 51 and the second electrode are, for example, metal materials. One of the first electrode 51 or the second electrode 52 functions as a drain electrode; and the other functions as a source electrode.

The graphene film 21 is provided between the first electrode 51 and the second electrode 52. The first electrode and the second electrode 52 electrically contact the graphene film 21. A current can be caused to flow between the first electrode 51 and the second electrode 52 via the graphene film 21.

A well (or a flow channel) 56 that is surrounded with a sidewall 55 is formed on the graphene film 21; and the ionic liquid 60 is contained inside the well 56. The probe molecule 31 that binds to the surface of the graphene film 21 is inside the ionic liquid 60.

Here, when the probe molecule 31 recognizes/captures the target substance, the electron state of the graphene changes due to the charge of the target substance because the target substance becomes proximal to the graphene surface. By electrically detecting this change, the existence and/or concentration of the target substance can be known. Although not illustrated, the surfaces of the first electrode 51 and the second electrode 52 are covered with an insulating film; and the surfaces of the first electrode 51 and the second electrode 52 do not have direct electrical conduction to the ionic liquid 60. The surface of the graphene film 21 also may be covered with an insulating body as necessary. For example, a phospholipid film, etc., may be used as the insulating body.

FIG. 2B is a schematic view showing the binding between the graphene film 21 and the probe molecule 31.

As the probe molecule 31, thiol-modified single strain DNA (deoxyribonucleic acid) is caused to bind to the amino-modified graphene film 21 via the linker 71.

The sensor of the embodiment includes the ionic liquid 60. Because the potential window of the ionic liquid 60 is wide, the voltage at which electrolysis occurs is high; and the potential of the sensor element 20 can be set broadly.

The viscosity can be changed by selecting the type of the ionic liquid 60. For example, by using an ionic liquid 60 having high viscosity, the ionic liquid 60 can be semi-immobilized at the desired position.

The vapor pressure of the ionic liquid 60 is low compared to that of an aqueous solution. Therefore, even in the case where the ionic liquid 60 is formed as a thin liquid layer in a thin film configuration, a wet state in which the ionic liquid 60 covers the probe molecule 31 can be maintained without drying. In such a case, the wet state also can be maintained by the ionic liquid 60 itself; and the wet state also can be realized by covering the probe molecule 31 with the ionic liquid 60 that includes moisture by utilizing the hygroscopicity (the capturing of moisture from the humidity of a vapor) of the ionic liquid 60.

By utilizing the hygroscopicity or the difficulty of drying of the ionic liquid 60, it is possible to maintain the probe molecule 31 constantly in a wet environment; and even the probe molecule 31 that does not have affinity (the ability of binding to the target substance) unless in the liquid phase or the probe molecule 31 that does not have affinity without moisture can be used.

In the case of a normal aqueous solution, for example, the aqueous solution undesirably dries instantaneously if the aqueous solution is set to be thin such as 1 mm or less. Conversely, it is also possible to maintain the ionic liquid 60 in a thin film state without drying even in the case where the ionic liquid 60 has a thickness of 1 mm or less, or even 1 μm or less.

Although a sensor that uses a graphene FET as the sensing portion is illustrated in FIG. 2A, the sensor may be a carbon nanotube FET or may be a charge detection element such as an ISFET (Ion sensitive FET) or the like, a surface plasmon resonance element, a SAW (Surface Acoustic Wave) element, a FBAR (Film Bulk Acoustic Resonator) element, a QCM (Quartz Crystal Microbalance) element, or a MEMS (Micro Electro Mechanical System) cantilever element.

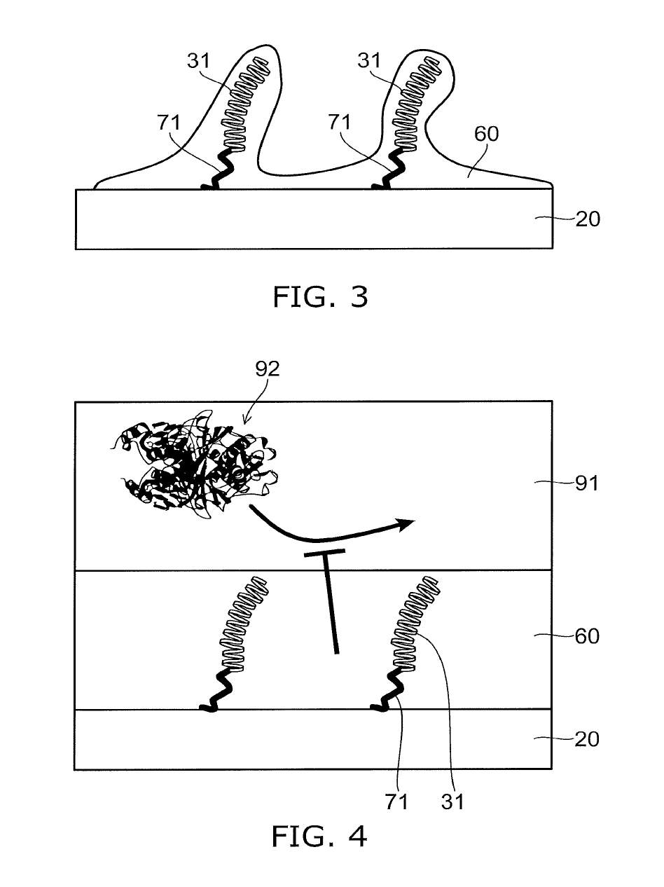

FIG. 3 is a schematic view of a sensor using the ionic liquid 60 that is a thin film.

The probe molecule 31 is covered with the thin film ionic liquid 60. In particular, the thin film state can be maintained by using the ionic liquid 60 having a high viscosity. Such a thin film ionic liquid 60 can perform highly-sensitive detection of a gas in the vapor phase such as a VOC (Volatile Organic Compound) by receiving the gas efficiently from the vapor phase. Because the ionic liquid 60 is the thin film, the VOC that dissolves from the vapor phase at the ionic liquid 60 surface instantaneously reaches the probe molecule 31 and can be detected with high sensitivity. Because the concentration of the VOC inside the thin film of the ionic liquid 60 is a high concentration, the association efficiency between the VOC and the probe molecule 31 also can be high.

Generally, it has been problematic that there are many hydrophobic molecules in VOCs that do not dissolve easily in an aqueous solution; and it has been difficult to increase the sensitivity; however, by using the ionic liquid 60 having a large hydrophobic group portion, the VOCs can be received more efficiently.

The sensor that uses such a thin film ionic liquid 60 can be used in quarantine, narcotic investigation, disease diagnosis, lifesaving in disasters, etc., as a sensor for sensing an ultra trace gas in air (a VOC, a toxic gas, a noxious gas, the odor of an illicit drug such as a narcotic, etc., the odor of food, a human, or an animal, or a low molecular-weight compound suggesting a disease).

Incidentally, the sense of smell of an animal detects odor components in the wet state by covering olfactory receptors with mucus having a high viscosity; but moisture is replenished constantly because the mucus undesirably dries quickly. In many cases, it is difficult to constantly supply moisture to an electronic component; but it is unnecessary to replenish moisture in the sensor using the ionic liquid 60.

In the case where an ionic liquid 60 that does not mix with an aqueous solution is used, the penetration of an enzyme can be prevented by using an ionic liquid 60 that does not have affinity with protein.

In such a case, by setting the ionic liquid 60 to be a thin film, it is easy for the target substance in the specimen aqueous solution to associate with the probe molecule 31 via the thin ionic liquid 60; and high sensing sensitivity is possible.

FIG. 4 is a schematic view of a sensor using an ionic liquid 60 that does not have affinity with protein.

According to such a sensor, it is possible to use an aptamer as the probe molecule 31 to test a biological liquid (blood, saliva, urine, etc.) 91.

Currently, an aptamer generally is not used to test a biological liquid. The reason is that doubts remain regarding the credibility of the inspection results because there is a possibility that the aptamer may be undesirably decomposed by a nuclease in the biological liquid.

Conversely, the aptamer (the probe molecule) 31 can be used even for a test using the biological liquid 91 including a nuclease (an enzyme) 92 due to the ionic liquid 60 blocking the penetration of the nuclease 92. Or, an ionic liquid 60 such that the enzyme activity of the nuclease does not function in the ionic liquid even if the nuclease enters the ionic liquid may be used.

By using the ionic liquid 60 having no affinity with the designated substance group, it is possible for the ionic liquid 60 to function as a blocking agent for a contaminant other than the substance which is the target. Misoperations of the sensor can be prevented by obstructing the penetration of a contaminant and/or a toxic substance that would deactivate the probe molecule 31.

Also, the specificity for the target substance can be increased by using the ionic liquid 60 having no affinity with the designated substance group. The target substance can be sorted by the probe molecule 31 while the ionic liquid 60 suppresses the penetration of a substance having no affinity with the ionic liquid 60. In such a case, the specificity increases because the target substance is sorted doubly by the affinity of the ionic liquid 60 and the substrate specificity of the probe molecule 31. As a result, for example, it is possible to increase the substrate specificity even in the case where a probe molecule 31 having a relatively small substrate specificity such as an olfactory receptor, lectin, a peptide oligomer, or the like is used. The variation of the substrate specificity also can be widened by changing the combination of the ionic liquid 60 and the probe molecule 31.

It is also possible to prevent the contaminant from becoming proximal to the probe molecule 31 by setting the layer of the ionic liquid 60 having the blocking function of the designated substance group to be thick.

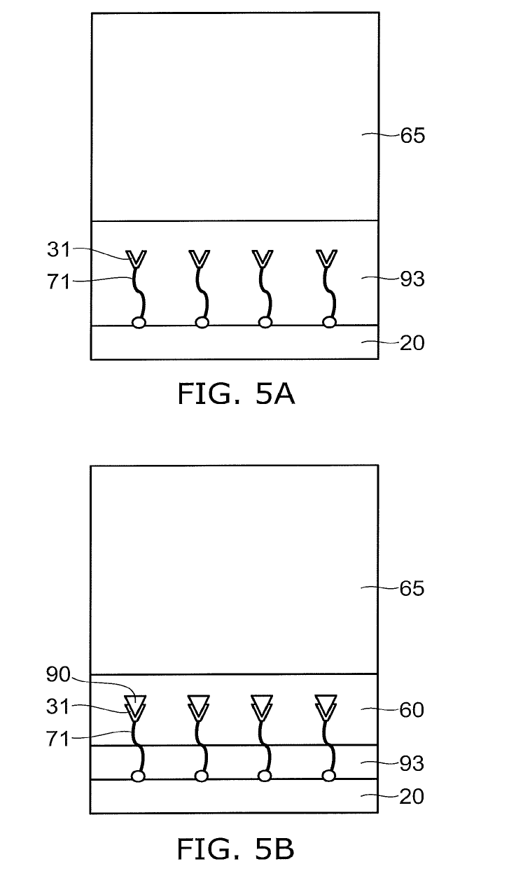

Normally, in many cases as shown in FIG. 5A, a blocking agent 93 is configured on the surface of the sensor element 20 to be thicker (higher) than the probe molecule 31. There is a risk that such a blocking agent 93 may also obstruct the association of the target substance and the probe molecule 31 and undesirably reduce the sensitivity. By using the ionic liquid 60 having affinity with the target substance but no affinity with the contaminant, the contaminant becoming proximal to the probe molecule 31 can be deterred without reducing the sensitivity. As a result, the SN ratio can be increased.

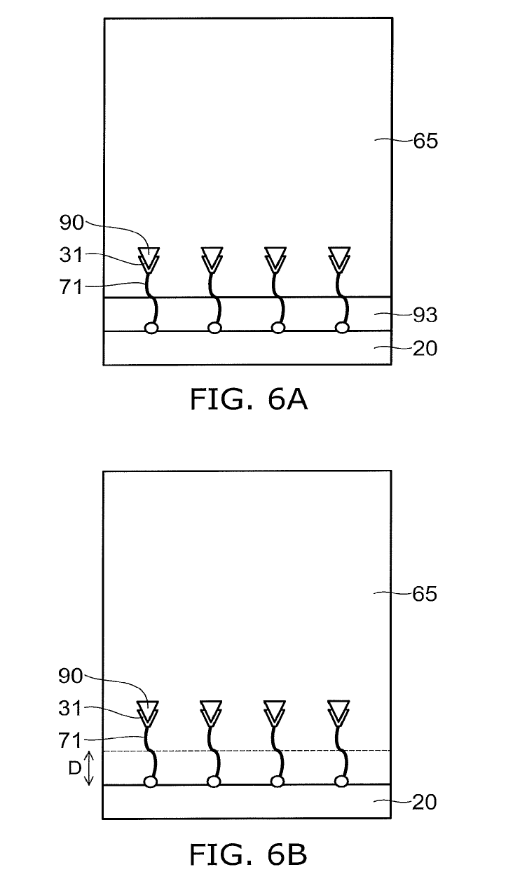

As shown in FIG. 5B, it is also possible to use the blocking agent (the blocking film) 93 that is thinner than the probe molecule 31 height to cover the surface of the sensor element 20 inside the ionic liquid 60. As shown in FIG. 6A, in the case where such a thin film blocking agent 93 is used even without using the ionic liquid 60, nonspecific adsorption due to the contaminant in an aqueous solution 65 can be prevented without reducing the sensitivity; and the SN ratio can be increased; but in the case where the ionic liquid 60 is used, the SN ratio can be increased further because a double effect of the blocking of the contaminant by the ionic liquid 60 and the prevention of the nonspecific adsorption of the contaminant by a blocking agent 63 is obtained.

The thickness of the electric double layer, i.e., the Debye length, can be changed by selecting the ionic liquid 60. In the ionic liquid 60, for example, as described in PNAS Jun. 11, 2013 vol. 110 no. 24 96 79, the Debye length may be thicker than the aqueous solution.

To detect the capture of the target substance by the probe molecule 31 as a charge, it is necessary for the target substance to be proximal inside (within the range of) the Debye length. This is because the displacement of the charge outside the Debye length D is undesirably shielded by the electric double layer as shown in FIG. 6B and therefore cannot be seen from the sensor element 20.

In the case where the ionic liquid 60 is used as shown in FIG. 7, compared to the case of FIG. 6B where the aqueous solution 65 is used, the Debye length D can be set to be thick; and, for example, it is possible to use a macromolecule such as a fragmented IgG antibody or enzyme as the probe molecule 31.

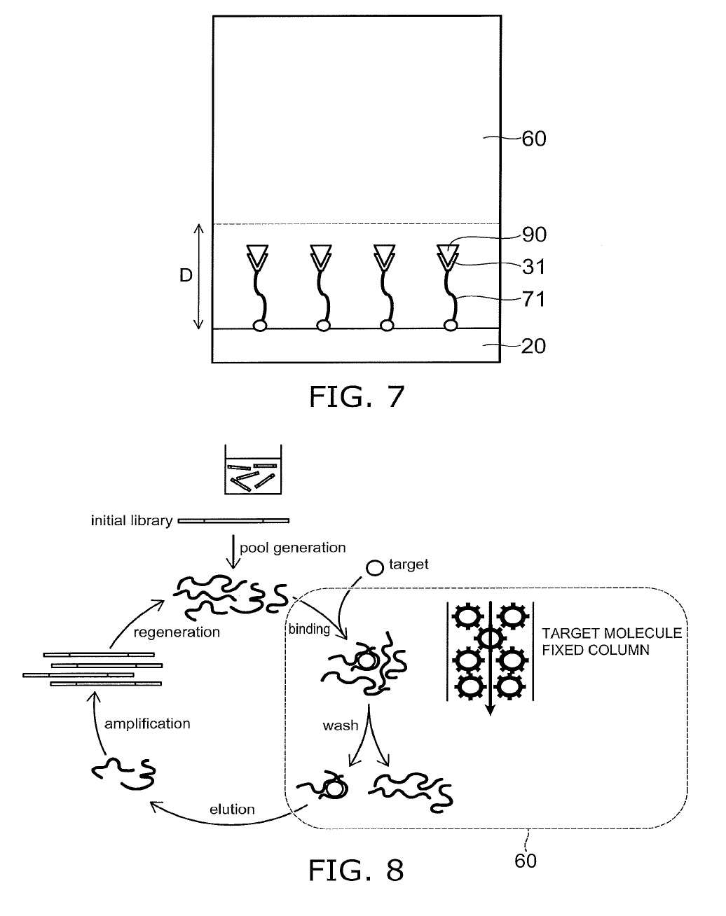

FIG. 8 is a schematic view showing a method for manufacturing an aptamer (a nucleic acid) as the probe molecule 31 having affinity in the ionic liquid 60 by using SELEX (Systematic Evolution of Ligands by EXponential enrichment). Multiple nucleic acids having different base sequences are mixed in the ionic liquid 60 with the designated substance (the target); a nucleic acid having a high association ability with the designated substance in the ionic liquid 60 is selected and recovered; and subsequently, amplification of the nucleic acid is performed in an aqueous solution by using PCR (Polymerase Chain Reaction). Only the nucleic acid having high binding strength to the designated substance in the ionic liquid 60 can be concentrated. By repeating such processes, the nucleic acid aptamer that has the strongest binding to the designated substance in the ionic liquid 60 can be discovered.

Although the amplification of the nucleic acid is performed using PCR in the example recited above, the amplification may be performed using other amplification methods, e.g., LAMP (Loop-Mediated Isothermal Amplification). Although the nucleic acid that is bound to the target is collected using a column in FIG. 8, it is also possible to use CE-SELEX (Capillary Electrophoresis Systematic Evolution of Ligands by Exponential enrichment) to separate by capillary electrophoresis by utilizing the difference between the charges of the nucleic acid bound to the target and the nucleic acid not bound to the target. In such a case, the nucleic acid that is in the ionic liquid 60 is separated by capillary electrophoresis.

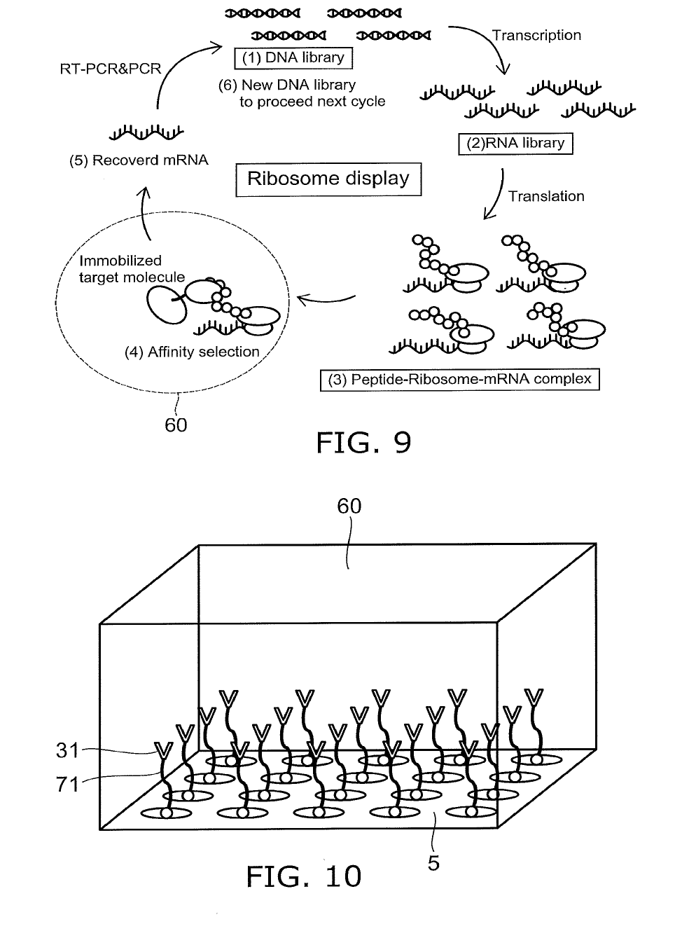

FIG. 9 is a schematic view showing a method for manufacturing a peptide aptamer as the probe molecule 31 having affinity in the ionic liquid 60 by using a two-hybrid method.

A polypeptide having different amino acid sequences is synthesized from multiple mRNA (messenger ribonucleic acid) having different base sequences by using a ribosome; and a complex in the state in which the mRNA, the ribosome, and the polypeptide are not separated is mixed with the designated substance (the target) into the ionic liquid 60.

Then, reverse transcription is performed for mRNA that is obtained by selecting and recovering the complex having the polypeptide having a high association ability with the designated substance in the ionic liquid 60; amplification is performed by PCR (Polymerase Chain Reaction); and a polypeptide is synthesized from the mRNA by using a ribosome.

FIG. 10 is a schematic view showing a method for manufacturing the probe molecule 31 having affinity in the ionic liquid 60.

The multiple probe molecules 31 made of multiple polypeptides having different amino acid sequences are immobilized in an array configuration on a substrate 5.

Then, the multiple probe molecules 31 that are immobilized on the substrate 5 are immersed in the ionic liquid 60 with the designated substance; and the designated probe molecule is selected by verifying the association ability between the designated substance and the multiple probe molecules 31 in the ionic liquid 60.

The multiple probe molecules 31 that are immobilized in an array configuration on the substrate 5 may be one of multiple nucleic acids having different base sequences or multiple antibodies having different paratopes.

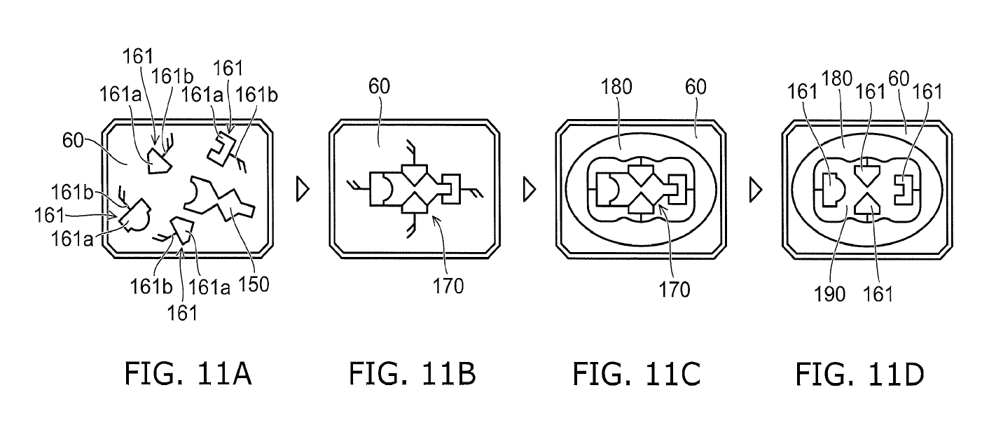

FIGS. 11A to 11D are schematic views showing a method for manufacturing a molecularly imprinted polymer having affinity in the ionic liquid 60.

As shown in FIG. 11A, a monomer molecule 161 and a target substance 150 that is used as a template are mixed into the ionic liquid 60. The monomer molecule 161 has a site 161a that binds to or associates with a characteristic portion of the target substance 150; and the monomer molecule 161 binds to or associates with the target substance 150 (FIG. 11B). Here, although the common numerals of 161 for the monomer molecule and 161a and 161b for the association sites are used in the description for convenience, multiple types of monomer molecules and association sites may be used.

Then, as shown in FIG. 11C, an association 170 of the template target substance 150 and the monomer molecule 161 is covered with a polymer 180; and polymerization is performed. The monomer molecule 161 binds to the polymer 180 and is immobilized by the site 161b binding to the polymer 180.

Subsequently, by extracting and removing the template target substance 150 while the monomer molecule 161 remains, the polymer 180 that has a cavity 190 where the target substance 150 can nicely fit is obtained (FIG. 11D). The monomer molecules 161 that can bind to or associate with the characteristic portions of the target substance 150 exist right at positions corresponding to the characteristic portions of the target substance 150 inside the cavity 190; therefore, the same substance as the target substance 150 used as the template binds or associates exceedingly specifically (selectively).

Although a method for sorting or manufacturing the probe molecule having affinity in the ionic liquid is shown in the example of the description recited above, it is also possible to use, in the ionic liquid, a probe molecule that has affinity in a normal aqueous solution.

In such a case, a liquid that has a high hygroscopicity may be used as the ionic liquid if moisture is necessary.

Even in the case of sorting or manufacturing the probe molecule having affinity in the ionic liquid, an ionic liquid that is hygroscopic may be used; and a probe molecule having affinity in a moisture-absorbing ionic liquid may be sorted or manufactured.

A reagent of an embodiment using the ionic liquid 60 will now be described.

As an example of the reagent, a fluorescent reagent that utilizes the principle of fluorescence resonance energy transfer (FRET), bioluminescence resonance energy transfer (BRET), or molecular beacon (MB) may be used.

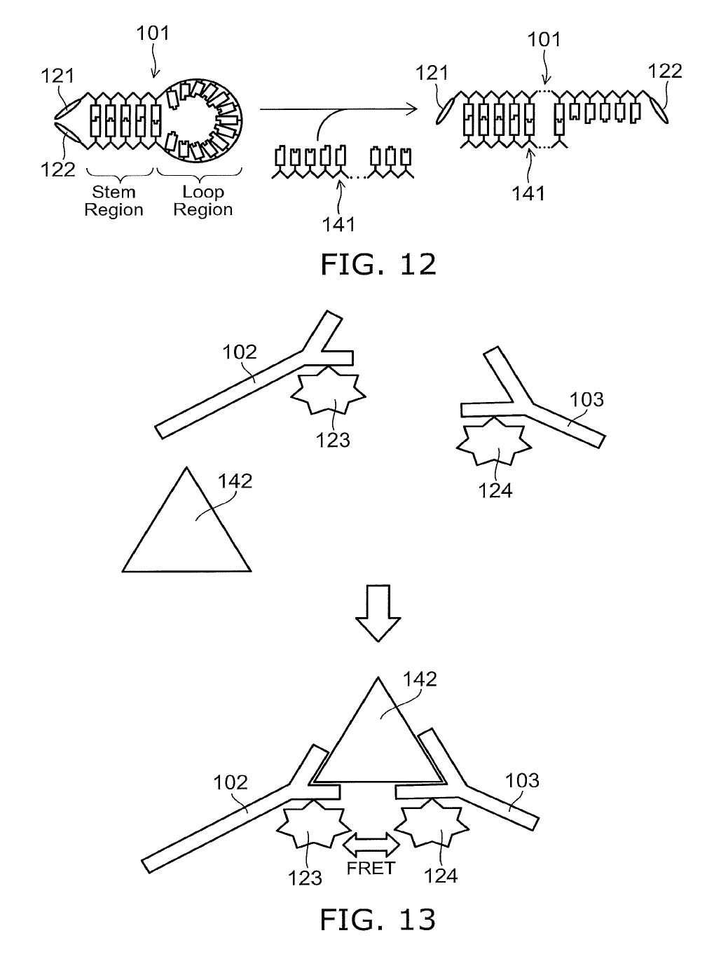

FIG. 12 is a schematic view of a reagent utilizing the MB principle.

The reagent includes an ionic liquid, a probe molecule 101 selectively associating with a designated substance in the ionic liquid, a first fluorescent dye 121, and a second fluorescent dye (or a quencher) 122.

The probe molecule 101 is, for example, a nucleic acid having a stem-loop (a hairpin loop) structure. The first fluorescent dye 121 binds to one terminal of the strand of the nucleic acid; and the second fluorescent dye (or the quencher) 122 binds to the other terminal.

The light emission wavelength of the first fluorescent dye 121 is shorter than the light emission wavelength of the second fluorescent dye 122. In the case where the light emission wavelength of the second fluorescent dye 122 is longer than the visible region, the second fluorescent dye 122 is a quencher because the light emission of the second fluorescent dye 122 cannot be seen. Or, a dark quencher that dissipates energy other than light such as heat, etc., may be used.

When the second fluorescent dye (or the quencher) 122 becomes proximal to the first fluorescent dye 121, resonance energy transfer occurs; and the fluorescence intensity of the first fluorescent dye 121 decreases. On the other hand, in the case where the second fluorescent dye 122 has a light emission characteristic in the visible region, even though the second fluorescent dye 122 is not excited by the excitation light for the first fluorescent dye 121, the second fluorescent dye 122 emits light due to the resonance energy transfer from the first fluorescent dye 121 to the second fluorescent dye 122. The distance between the first fluorescent dye 121 and the second fluorescent dye (or the quencher) 122 changes due to the structural change of the probe molecule 101 associating with the designated substance.

When a target gene 141 exists as the designated substance in the ionic liquid, the nucleic acid (the probe molecule) 101 binds to the target gene 141; the structure is no longer a loop structure; and the two terminals of the strand separate.

Here, in the case where the first fluorescent dye 121 binds to one terminal of the strand and the quencher (the quenching group) 122 binds to the other terminal, the distance between the first fluorescent dye 121 and the quencher 122 becomes large due to the binding of the target gene 141; and the light emission intensity of the first fluorescent dye 121 increases because the resonance energy transfer stops.

On the other hand, in the case where the first fluorescent dye 121 and the second fluorescent dye 122 bind to the two terminals of the strand, the distance between the first fluorescent dye 121 and the second fluorescent dye 122 becomes large due to the binding of the target gene 141; because the resonance energy transfer stops, the light emission intensity of the first fluorescent dye 121 increases; and the light emission from the second fluorescent dye 122 is quenched.

FIG. 13 is a schematic view of a reagent utilizing the FRET/BRET principle.

The reagent includes an ionic liquid, the first probe molecule (the antibody or the like) 102 that selectively associates with a designated substance (a target substance) 142 in the ionic liquid, a first fluorescent dye 123, a second probe molecule (an antibody or the like) 103 that associates in the ionic liquid at a site of the designated substance 142 that is different from a site where the first probe molecule 102 associates, and a second fluorescent dye (or a quencher) 124.

The light emission wavelength of the first fluorescent dye 123 is shorter than the light emission wavelength of the second fluorescent dye 124. In the case where the light emission wavelength of the second fluorescent dye 124 is longer than the visible region, the second fluorescent dye 124 is a quencher because the light emission of the second fluorescent dye 124 cannot be seen. Or, a dark quencher that dissipates energy other than light such as heat, etc., may be used.

The first fluorescent dye 123 binds to the first probe molecule 102; and the second fluorescent dye (or the quencher) 124 binds to the second probe molecule 103. By the first probe molecule 102 and the second probe molecule 103 associating with the designated substance 142, the first fluorescent dye 123 and the second fluorescent dye (or the quencher) 124 become proximal. The fluorescence intensity of the first fluorescent dye 123 decreases due to the resonance energy transfer because the first fluorescent dye 123 and the second fluorescent dye (or the quencher) 124 are proximal.

In the case where the quencher 124 binds to the second probe molecule 103, the first fluorescent dye 123 and the quencher 124 become proximal due to the first probe molecule 102 and the second probe molecule 103 associating with the designated substance 142; and the light emission intensity of the first fluorescent dye 123 weakens. The existence and/or the concentration of the designated substance 142 can be known from the light emission intensity of the first fluorescent dye 123 because the quencher 124 does not emit light regardless of the distance to the first probe molecule 102.

In the case where the second fluorescent dye 124 binds to the second probe molecule 103, the first fluorescent dye 123 and the second fluorescent dye 124 become proximal due to the first probe molecule 102 and the second probe molecule 103 associating with the designated substance 142; the light emission intensity of the first fluorescent dye 123 weakens; and the second fluorescent dye 124 emits light. At this time, because the second fluorescent dye 124 is excited by the resonance energy transfer from the first fluorescent dye 123, the second fluorescent dye 124 emits light even though the second fluorescent dye 124 is not excited by the light exciting the first fluorescent dye 123. Thereby, the existence and/or the concentration of the designated substance 142 can be known.

Although the description recited above is an example of FRET, it is also possible to use a bioluminescent dye instead of the first fluorescent dye 123. In such a case, it is unnecessary to irradiate the excitation light because the bioluminescent dye is excited/emits light due to a chemical reaction. Even in such a case, the resonance energy transfer with the second fluorescent dye (or the quencher) 124 occurs. This configuration is called BRET.

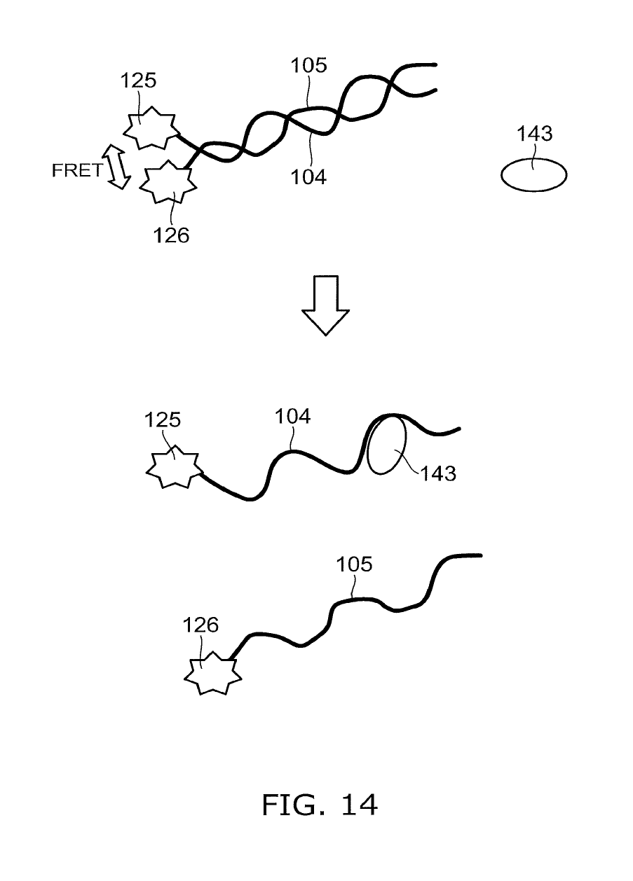

FIG. 14 is a schematic view of a reagent utilizing the FRET/BRET principle.

The reagent includes an ionic liquid, a first probe molecule (an aptamer) 104 that selectively associates with a designated substance (a target substance) 143 in the ionic liquid, a first fluorescent dye 125, a second probe molecule (an aptamer) 105 that associates with the first probe molecule 104 in the ionic liquid, and a second fluorescent dye (or a quencher) 126.

The light emission wavelength of the first fluorescent dye 125 is shorter than the light emission wavelength of the second fluorescent dye 126. In the case where the light emission wavelength of the second fluorescent dye 126 is longer than the visible region, the second fluorescent dye 126 is a quencher because the light emission of the second fluorescent dye 126 cannot be seen. Or, a dark quencher that dissipates energy other than light such as heat, etc., may be used.

In the example shown in FIG. 14, the first fluorescent dye 125 binds to the tip of the first probe molecule (the aptamer) 104; and the second fluorescent dye 126 binds to the tip of the second probe molecule (the aptamer) 105. Due to the first probe molecule 104 associating with the designated substance 143, the first probe molecule 104 and the second probe molecule 105 dissociate; the distance between the first fluorescent dye 125 and the second fluorescent dye 126 increases; and the light emission intensity of the first fluorescent dye 125 increases.

In the case where the quencher 126 binds to the second probe molecule 105, the distance between the first fluorescent dye 125 and the quencher 126 increases due to the first probe molecule 104 and the second probe molecule 105 associating with the designated substance 143; therefore, the light emission intensity of the first fluorescent dye 125 that had been reduced by the resonance energy transfer increases. The existence and/or the concentration of the designated substance 143 can be known from the light emission intensity of the first fluorescent dye 125 because the quencher 126 does not emit light regardless of the distance to the first probe molecule 104.

In the case where the second fluorescent dye 126 binds to the second probe molecule 105, the distance between the first fluorescent dye 125 and the second fluorescent dye 126 increases due to the first probe molecule 104 and the second probe molecule 105 associating with the designated substance 143; therefore, the light emission intensity of the first fluorescent dye 125 increases; and the second fluorescent dye 126 is quenched. Thereby, the existence and/or the concentration of the designated substance 143 can be known.

Although the description recited above is an example of FRET, it is also possible to use a bioluminescent dye instead of the first fluorescent dye 125. In such a case, it is unnecessary to irradiate the excitation light because the bioluminescent dye is excited/emits light due to a chemical reaction. Even in such a case, the resonance energy transfer with the second fluorescent dye (or the quencher) 126 occurs. This configuration is called BRET.

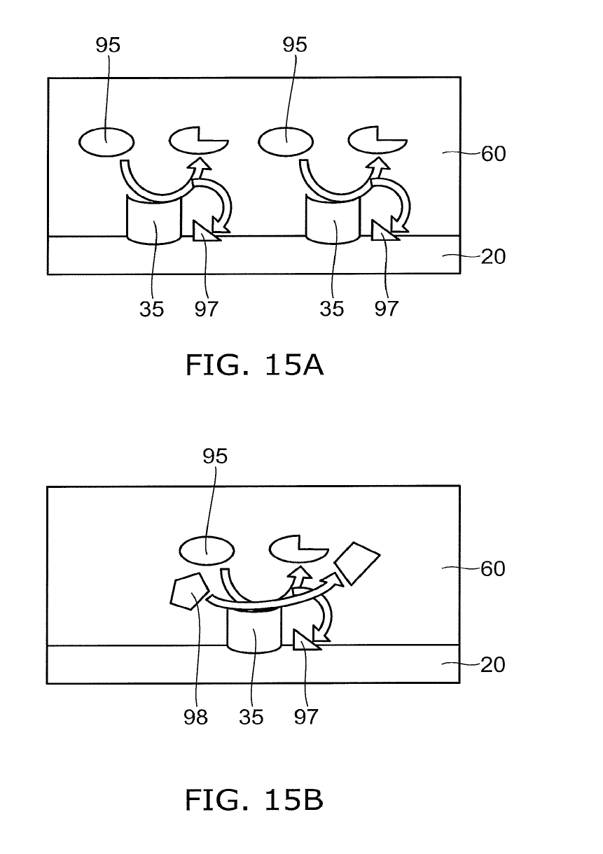

FIG. 15A is a schematic view of a sensor including a catalyst molecule that promotes a designated chemical reaction in the ionic liquid 60.

As the catalyst molecule, an enzyme 35 that is active in the ionic liquid 60 is disposed on the surface of the sensor element 20. The ionic liquid 60 covers the enzyme 35 and the surface of the sensor element 20 and shields the enzyme 35 from the external environment (liquid or vapor).

A substrate 95 is converted into another substance by an enzyme reaction in the ionic liquid 60. The sensor element 20 electrically or optically detects a chemical substance 97 produced by or increasing or decreasing due to the enzyme reaction. Or, in the case where oxidation reduction accompanies the chemical reaction, the increase or decrease of the charge accompanying the chemical reaction is detected.

For example, the ionic liquid 60 is 1-butyl-2,3-dimethylimidazolium polyethylene glycol hexadecyl ether sulphate; the enzyme 35 is a lipase; and the substrate 95 is secondary alcohol.

As shown in FIG. 15B, a coenzyme 98 may be added as necessary. In such a case, it is also possible to electrically or optically detect the chemical change of the coenzyme. Or, a catalyst molecule may be used instead of the enzyme 35. As the catalyst molecule, for example, a metal, a metal oxide, etc., may be used.

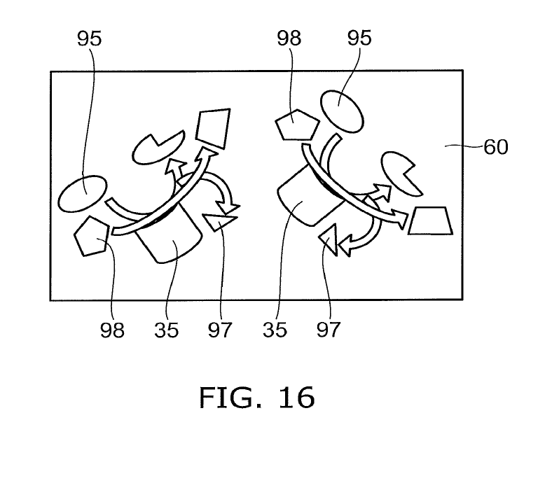

FIG. 16 is a schematic view of a designated substance removal reagent including the enzyme 35 that promotes a designated chemical reaction in the ionic liquid 60, and a designated substance removal device including the reagent.

For example, the formaldehyde-decomposing enzyme FALDH (formaldehyde dehydrogenase) is used as the enzyme 35. A coenzyme also is used as necessary. In the case of FALDH, a coenzyme NAD⁺ (Nicotinamide Adenine Dinucleotide) is necessary to assist the formaldehyde decomposition reaction and is therefore added. These enzyme and/or coenzymes are surrounded with the ionic liquid 60; and a wet environment is maintained. In the case where the ionic liquid 60 also is hygroscopic, moisture is acquired from the vapor phase of the external environment; and a state including moisture is maintained.

The enzyme 35 decomposes a noxious gas (aldehyde, ammonia, etc.) into an innocuous molecule in the ionic liquid 60. For example, in the case of FALDH, formaldehyde which is a cause of sick house syndrome is converted into formic acid by oxidization. At this time, the coenzyme NAD⁺ is reduced into NADH. FDH (formate dehydrogenase) is added in the case where the pungent odor of formic acid is a problem. FDH converts formic acid into carbon dioxide by oxidization. At this time, the coenzyme NAD⁺ is reduced into NADH.

By mounting the designated substance removal reagent on a sensor element, the existence and/or the concentration of the designated substance can be known; and the progress of the chemical reaction also can be known. Although an enzyme is used as the catalyst material in the description of the example, a chemical substance that has catalytic activity may be used; and it is also possible to use, for example, a substance such as a metal or a metal oxide.

It is also possible to use a constituent ion of the ionic liquid 60 as the probe molecule.

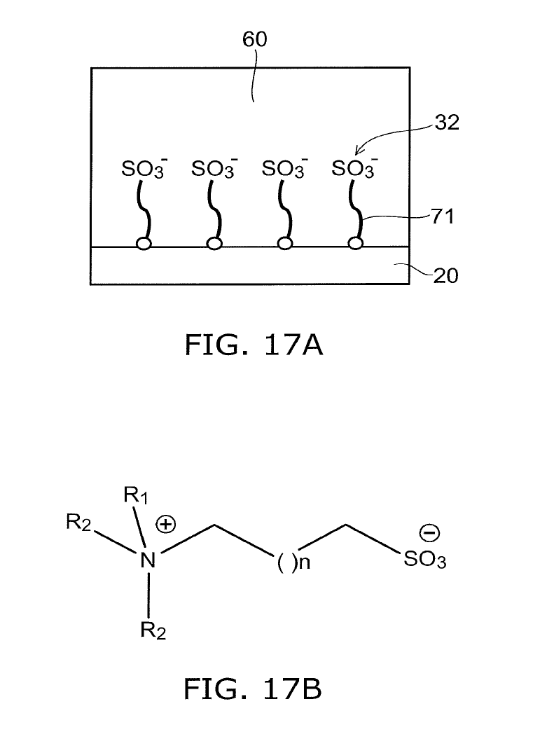

FIG. 17A is a schematic view of a sensor in which a constituent ion of the ionic liquid 60 is embedded as a probe molecule 32.

FIG. 17B is a structural formula of an example of the ionic liquid 60 used in FIG. 17A.

An ion that is included in the ionic liquid 60 binds to the surface of the sensor element 20 and functions as the probe molecule 32. The target substance can be selected by the ionic liquid that is used; and it is unnecessary to manufacture many types of devices having different probe molecules to match the target substances. It is possible for the constituent ion of the ionic liquid 60 to bind to the surface of the sensor element 20 self-aligningly.

According to the above-described embodiment, by using an ionic liquid, it is possible to prevent a decrease in function of a probe molecule or a catalyst molecule due to drying of a liquid, and to maintain a detection ability and detection accuracy of a target substance over a long period of time.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modification as would fall within the scope and spirit of the inventions.

What is claimed is:

1. A sensor, comprising:
an ionic liquid;
a probe molecule selectively associating with a designated substance in the ionic liquid; and
a sensor element detecting an association of the probe molecule with the designated substance.

2. The sensor according to claim 1, wherein the probe molecule includes at least one selected from the group consisting of an antibody, an aptamer, a peptide aptamer, an enzyme, and a molecularly imprinted polymer.

3. The sensor according to claim 1, wherein a liquid surface height of the ionic liquid is higher than an upper end height of the probe molecule when referenced to a surface of the sensor element.

4. A sensor, comprising:
an ionic liquid;
a catalyst molecule promoting a designated chemical reaction in the ionic liquid; and
a sensor element detecting at least one of a chemical substance or a charge, the at least one increasing or decreasing due to the chemical reaction.

5. The sensor according to claim 1, wherein the sensor element is a charge detection element, a surface plasmon resonance element, a SAW (Surface Acoustic Wave) element, a FBAR (Film Bulk Acoustic Resonator) element, a QCM (Quartz Crystal Microbalance) element, or a MEMS (Micro Electro Mechanical System) cantilever element.

6. The sensor according to claim 5, wherein the charge detection element includes graphene or carbon nanotubes.

7. A reagent, comprising:
an ionic liquid;
a first probe molecule selectively associating with a designated substance in the ionic liquid;
a first fluorescent dye; and
at least one of a second fluorescent dye or a quencher,
due to the first probe molecule associating with the designated substance, a distance between the first fluorescent dye and the at least one of the second fluorescent dye or the quencher changes, and a fluorescence intensity due to a resonance energy transfer changes.

8. The reagent according to claim 7, wherein the first fluorescent dye and the at least one of the second fluorescent dye or the quencher bind to the first probe molecule.

9. The reagent according to claim 7, further comprising a second probe molecule associating in the ionic liquid with a site of the designated substance different from a site of the designated substance where the first probe molecule associates,
the first fluorescent dye binding to the first probe molecule,
the at least one of the second fluorescent dye or the quencher binding to the second probe molecule,
the first fluorescent dye and the at least one of the second fluorescent dye or the quencher becoming proximal due to the first probe molecule and the second probe molecule associating with the designated substance.

10. The reagent according to claim 7, further comprising a second probe molecule associating with the first probe molecule in the ionic liquid,
the first fluorescent dye binding to one of the first probe molecule or the second probe molecule,
the at least one of the second fluorescent dye or the quencher binding to the other of the first probe molecule or the second probe molecule,
due to the first probe molecule associating with the designated substance, the first probe molecule and the second probe molecule dissociate, and a distance between the first fluorescent dye and the at least one of the second fluorescent dye or the quencher increases.

11. A method for manufacturing a probe molecule, comprising:
mixing a plurality of nucleic acids having different base sequences with a designated substance in an ionic liquid; and
obtaining an aptamer associating in the ionic liquid with the designated substance by performing amplification in an aqueous solution after selecting and recovering the nucleic acid having associated with the designated substance.

12. The method according to claim 11, wherein the aptamer associating in the ionic liquid with the designated substance is obtained by repeating, after the amplification in the aqueous solution, processes of mixing in an ionic liquid with the designated substance, selecting and recovering the nucleic acid having associated with the designated substance, and performing amplification in an aqueous solution.

13. The method according to claim 11, wherein the amplification of the nucleic acid is performed by PCR (Polymerase Chain Reaction) or LAMP (Loop-Mediated Isothermal Amplification).

14. The method according to claim 11, wherein the nucleic acid having associated with the designated substance in the ionic liquid is recovered using a column fixing the designated substance.

15. The method according to claim 11, wherein an associated substance of the designated substance and the nucleic acid is recovered using capillary-electrophoresis.

16. The method according to claim 11, wherein the nucleic acid is DNA.

17. The method according to claim 11, wherein
the nucleic acid is RNA,
reverse transcription is performed before the amplification of the nucleic acid, and
transcription is performed after the amplification.

18. A method for manufacturing a probe molecule, comprising:
using a ribosome to synthesize a polypeptide having different amino acid sequences from a plurality of mRNA (messenger RNA) having different base sequences;
mixing, with a designated substance into an ionic liquid, a complex in which the mRNA, the ribosome, and the polypeptide are not separated; and
obtaining a peptide aptamer associating with the designated substance by selecting and recovering the complex having associated at a polypeptide site with the designated substance, subsequently extracting mRNA and performing reverse transcription into DNA, performing amplification of the DNA using PCR (Polymerase Chain Reaction) or LAMP (Loop-Mediated Isothermal Amplification), performing transcription into mRNA, subsequently repeating processes of synthesizing a polypeptide, mixing with the designated substance in an ionic liquid, selecting and recovering an associated substance, performing reverse transcription, performing amplification, and performing transcription, and subsequently synthesizing a polypeptide from the mRNA by using a ribosome.

19. A method for manufacturing a probe molecule, comprising:
immobilizing a plurality of probe molecules in an array configuration on a substrate, the plurality of probe molecules including at least one type selected from the group consisting of a plurality of nucleic acids having different base sequences, a plurality of polypeptides having different amino acid sequences, and a plurality of antibodies having different paratopes; and
selecting a designated probe molecule by immersing, in an ionic liquid with a designated substance, the plurality of probe molecules immobilized on the substrate and by verifying an association ability of the plurality of probe molecules and the designated substance in the ionic liquid.

20. A method for manufacturing a polymer molecule associating with a designated substance, comprising:
mixing the designated substance and a monomer molecule in an ionic liquid, the monomer molecule having a site associating with the designated substance in the ionic liquid and a second reaction site;
polymerizing a polymer precursor molecule on an associated substance of the designated substance and the monomer molecule after the monomer molecule has associated with the designated substance, the polymer precursor molecule having a site binding to the second reaction site of the monomer molecule, the polymer precursor molecule becoming a polymer by polymerization; and
subsequently removing the designated substance.
